(19) United States
(12) Patent Application Publication
(10) Pub. No.: US 2005/0033307 A1
(43) Pub. Date: Feb. 10, 2005

(54) Locking surgical instrument

(76) Inventors: Michael A. Cook, Claypool, IN (US); Rodney L. Bays, Pierceton, IN (US); Billy N. Sisk, Claypool, IN (US); Karthik N. Suryanarayan, Warsaw, IN (US)

Correspondence Address: ZIMMER TECHNOLOGY - REEVES, P. O. BOX 1268, ALEDO, TX, 76008, US

(21) Appl. No.: 10/634,967

(22) Filed: Aug. 5, 2003

(51) Int. Cl. A61B 17/58

(52) U.S. Cl. 606/104

(57) ABSTRACT

The present invention provides a locking surgical instrument for gripping a surgical workpiece.

### BACKGROUND

During surgical operations it is often necessary to manipulate a workpiece, such as an implant or trial implant, by imparting axial and/or torsional forces on the workpiece. Manipulating these workpieces can be difficult due to small size of the workpiece, the depth and/or narrowness of the surgical wound, and/or the need to impart large forces. For example, it is often necessary to insert and remove pins and screws from surgical sites.

### SUMMARY

The present invention provides a locking surgical instrument for gripping a surgical workpiece.

In one aspect of the invention, a locking surgical instrument for gripping a surgical workpiece includes a body with a shaft having a first end, a second end, and an axis therebetween, an engagement tip formed adjacent the second end being receivable by a workpiece, a portion of the engagement tip being divided by one or more slits from the second end toward the first end for a predetermined distance to form a plurality of segments able to be biased radially outwardly to grip the workpiece in a force transmitting relationship; and an actuator engageable with the body such that movement of the actuator biases the segments to grip the workpiece.

In another aspect of the invention, a locking surgical instrument for gripping a pin of a knee prosthesis includes a body with an elongated shaft having a first end, a second end, and an axis therebetween, a polygonal engagement tip formed adjacent the second end being receivable by a similarly shaped opening on the pin, a portion of the engagement tip being slit from the second end toward the first end for a predetermined distance to divide the tip into a plurality of segments able to be biased radially outwardly to grip the workpiece in axial force transmitting relationship, the body shaft being axially cannulated from the first end toward the second end such that the cannula extends under the slit portion a predetermined amount; and an actuator including a shaft having a first end, a second end, and an axis therebetween, the shaft being receivable within the cannula for axial translation between an unlocked position in which the actuator second end is spaced from the body shaft second end and a locked position in which the actuator second end is nearer the body shaft second end and biases the segments.

In another aspect of the invention, a method of gripping a surgical workpiece includes providing an instrument having a shaft with an axis and an engagement tip formed at one end, a portion of the engagement tip being slit to divide the tip into a plurality of segments able to be biased radially outwardly, and an actuator engageable with the body such that movement of the actuator biases the segments outwardly; inserting the engagement end into an opening in the surgical workpiece; and moving the actuator to bias the segments outwardly to grip the workpiece in axial force transmitting relationship.

### BRIEF DESCRIPTION OF THE DRAWINGS

Various embodiments of the present invention will be discussed with reference to the appended drawings. These drawings depict only illustrative embodiments of the invention and are not to be considered limiting of its scope.

FIG. 1 is a front section view of a surgical instrument according to the present invention with a workpiece;

FIG. 2 is a detailed side elevation view oriented along line 2-2 of FIG. 1;

FIG. 3 is a detailed front section view of the end of the surgical instrument of FIG. 1;

FIG. 4 is an alternative arrangement of the engagement end of the surgical instrument of FIG. 1;

FIG. 5 is an alternative arrangement of the engagement end of the surgical instrument of FIG. 1;

FIG. 6 is an alternative arrangement of the engagement end of the surgical instrument of FIG. 1;

FIG. 7 is a detail side elevation view similar to FIG. 2 with the actuator fully engaged and no workpiece present;

FIG. 8 is a front section view of the surgical instrument of FIG. 1 assembled with a workpiece; and

FIG. 9 is a perspective view of the surgical instrument of FIG. 1 in engagement with a knee hinge post extension.

### DESCRIPTION OF THE ILLUSTRATED EMBODIMENTS

Embodiments of a locking surgical instrument include an instrument body having an engagement tip for engaging a workpiece in force transmitting relationship. The workpiece may include an articular component, stem, shaft, pin, screw, and/or other appropriate workpiece. For example, the workpiece may be a hinge pin or a hinge post extension of a rotating hinge knee.

The body may include a shaft with a handle at one end and an engagement tip at the other end. The shaft may be sufficiently elongated to extend into a deep wound or into a surgical assembly. The handle may include an enlarged knob and may further include knurling, scallops, T-handles, and/or other suitable features to enhance a user's grip on the handle. The engagement tip may grip the workpiece in axial and/or torsional force transmitting relationship. Force transmission may be achieved by frictional and/or positive engagement of the engagement end and workpiece. The engagement tip may be circular or non-circular, including polygonal, elliptical, star-shaped, and/or other suitable shape for engaging the workpiece. Examples of polygonal engagement shapes include the triangle, quadrilateral, pentagon, hexagon, heptagon, octagon, etc. The engagement tip may be slit one or more times to form a plurality of segments able to be biased radially outwardly to grip the workpiece. The slits may be positioned to avoid the vertices, if present, of the engagement tip such that the vertices are intact and able to bite into the workpiece when the segments are biased outwardly. The body shaft may be cannulated to receive an actuator for biasing the engagement tip segments outwardly.

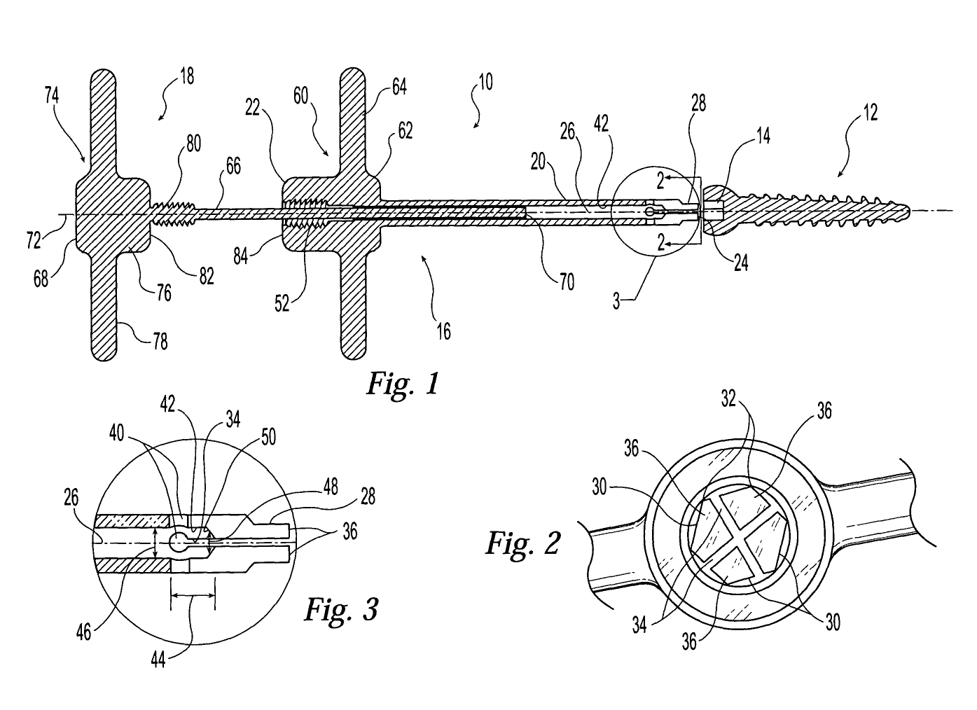

FIGS. 1-9 depict an illustrative embodiment of a locking surgical instrument 10 for engaging a workpiece 12 such as a screw having a female engagement 14. In the embodiment depicted in FIG. 1, the female engagement 14 is hex-shaped; however, other shapes are contemplated and fall within the scope of the present invention. The instrument 10 includes a body 16 and an actuator 18. The body 16 includes a shaft 20 having a first end 22, a second end 24, and an axis 26 from the first end to the second end. An engagement tip 28 is formed adjacent the second end 24. The engagement tip 28 matingly engages the female engagement 14 of the workpiece 12.

In the illustrative embodiment of FIGS. 1-4, the engagement tip 28 includes a hexagonal cross section with flats 30 and vertices 32. The engagement tip 28 is slit from the second end 24 toward the first end 22 for a predetermined distance to form a plurality of segments 36 able to be biased outwardly to grip the workpiece 12 in force transmitting relationship. In the illustrative embodiment, two slits 34 pass through the engagement tip 28 to form four segments 36. The slits 34 may pass through the tip 28 at any orientation according to the present invention, including through the vertices 32. However, it is believed, by the present inventors, to be advantageous to avoid the vertices 32 such that they remain intact. The vertices 32 present an edge that can bite into the corresponding vertices of the workpiece female engagement 14. The illustrative engagement tip 28 is divided by perpendicular slits 34, rotated to miss the vertices 32, into four asymmetrical segments 36. Alternatively, one, three, or some other number of slits may divide the tip. For example, two slits oriented at 120° to one another may divide the tip symmetrically into two pairs of opposing like-shaped segments. Also, for example, one slit across opposing flats or vertices may divide the tip into two symmetric segments.

In the illustrative embodiment, the slits 34 terminate with a circular stress relieving opening 40. As the segments 36 are biased outwardly, the end of each slit 34 is strained resulting in stress in the material surrounding the end of the slit 34. By having an opening 40 with a radius greater than one-half the width of the slit 34, the strain acts over a larger area than it would for a sharp or a full radius and the stresses are lower. On the other hand it is advantageous to maximize the contact area between the engagement tip 28 and the workpiece 12 to increase the torque transfer capacity of the junction. The illustrated arrangement obtains the benefits of a large slit terminus to reduce stresses and a small slit width to maximize the engagement area of the engagement tip 28.

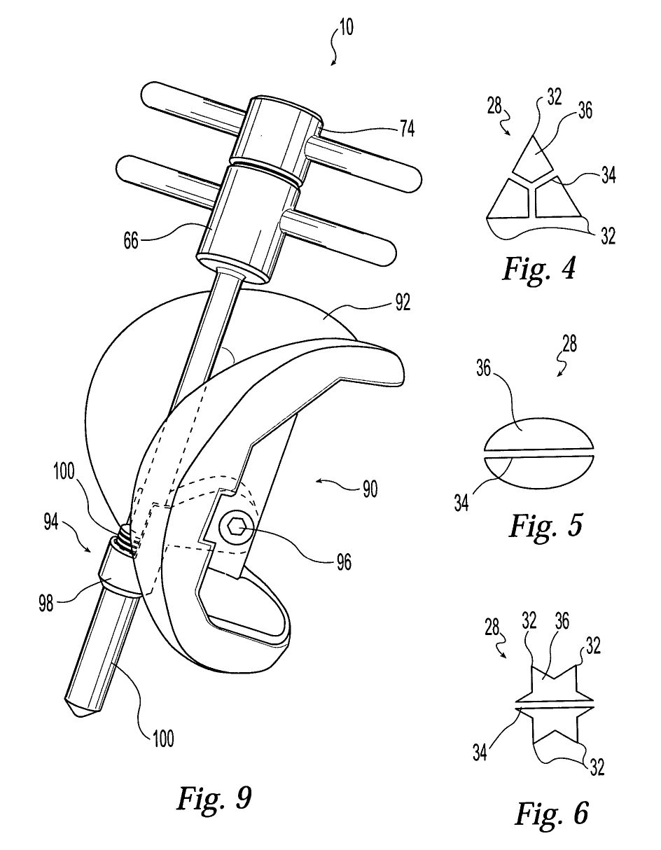

FIGS. 4-6 illustrate alternative exemplary tip configurations. FIG. 4 depicts a triangular engagement tip 28 divided into three symmetrical segments 36 by three slits 34. The slits 34 bisect the flats 30 and meet at the axis. FIG. 5 depicts an elliptical tip 28 divided into two segments 36 by a single slit 34. FIG. 6 depicts a star-shaped tip 28 divided into two segments 36 by a single slit 34 through two of the six vertices 32.

The body shaft 20 is axially cannulated 42 from the first end 22 toward the second end 24 such that the cannula 42 extends into the slit 34 portion of the engagement tip 28 a predetermined distance 44. The cannula 42 has a first diameter 46 toward the first end 22 and a second diameter 48 within the slit 34 portion that is smaller than the first diameter 46. The change in diameter allows the actuator 18 to extend through the first diameter to the second diameter 48 where it acts to bias the segments 36. The cannula 42 includes a tapered surface portion 50 that transitions from the first diameter 46 to the second diameter 48. Female threads 52 are formed around the cannula 42 adjacent the first end 22.

A handle 60 is formed adjacent the first end 22. The handle 60 includes a knob 62. A “T”-handle 64 is further incorporated into the handle 60 by forming radially extending arms on the knob. The “T”-handle allows greater torsional and axial force input by the user. Any portion of the handle 60 may be textured or shaped to further enhance the grip such as by grit blasting, knurling, scalloping, or other suitable means.

The actuator 18 includes a shaft 66 having a first end 68, a second end 70, and an axis 72 between the first and second ends. A handle 74 is formed adjacent the first end 68. The handle 74 includes a knob 76. A “T”-handle 78 is further incorporated into the handle 74 by forming radially extending arms on the knob. The “T”-handle allows greater torsional and axial force input by the user. Any portion of the handle 74 may be textured or shaped to further enhance the grip such as by grit blasting, knurling, scalloping, or other suitable means. Male threads 80 are formed about the shaft 66 adjacent the knob 76.

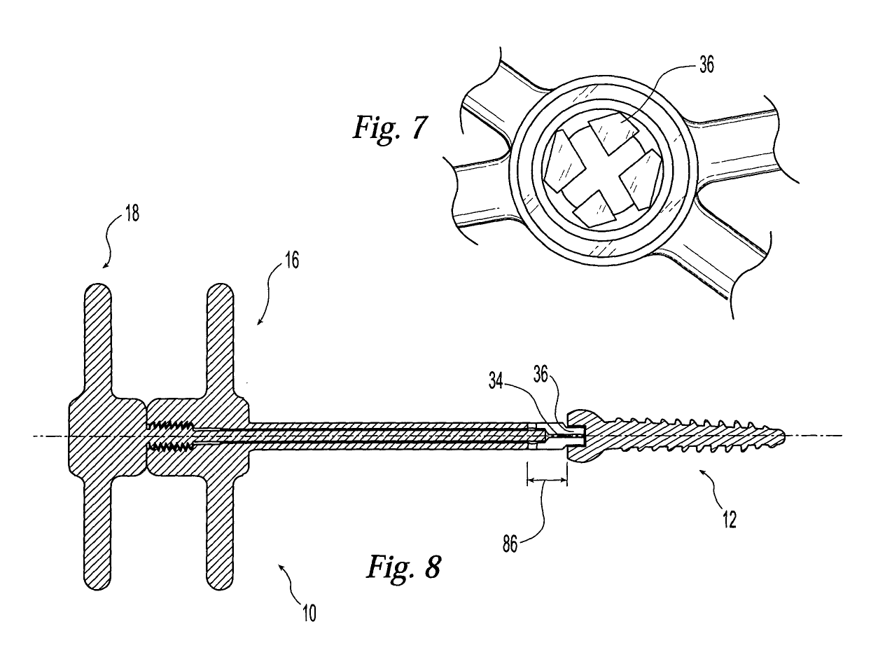

In use, the actuator shaft 66 is positioned within the cannula 42 and the actuator threads 80 engage the body threads 52. Threading the actuator 18 into the body 16 causes the actuator second end 70 to advance toward the cannula second diameter 48. Once the second end 70 contacts the tapered surface 50, further forward movement of the actuator 18 biases the segments 36 radially outwardly. Alternatively, the actuator 18 second end may be tapered to engage the second diameter 48. FIG. 7 depicts how the segments 36 move outwardly when biased by the actuator 18 and not constrained by a workpiece. The distance between the actuator second end 70 and the face 82 of the actuator knob 76 is controlled relative to the distance between the tapered surface 50 and the face 84 of the body knob 62. This permits a predetermined amount of engagement and biasing of the segments 36. Advantageously, the amount of engagement is within the elastic limits of the segments 36 such that even if the actuator 18 is fully seated without a workpiece in place, the segments 36 will undergo no permanent deformation and will return to their original position when the actuator 18 is withdrawn. Furthermore, it is convenient if the threads 52, 80 and faces 82, 84 are positioned such that the “T”-handles 64,78 of the body 16 and actuator 18 are near alignment with one another when the faces 82, 84 abut.

FIG. 8 depicts the instrument 10 engaged with a workpiece 12. The actuator 18 is engaged to bias the segments 36 radially into engagement with the workpiece 12 to enable the transmission of axial forces and torque to the workpiece. Further engagement of the actuator 18 increases radial engagement forces. The slits 34 extend beyond the workpiece a predetermined distance 86 to allow the segments 36 to bow radially outwardly to maintain radial spring tension in the engagement and to prevent breakage of the instrument 10 or workpiece 12. The predetermined distance 86 along with the predetermined amount of engagement of the actuator 18 and tapered surface 50 are advantageously controlled such that bowing of the segments 36 is within their elastic limits. Thus, even if the actuator 18 is fully seated with a tight fitting workpiece in place, the segments 36 will undergo no permanent deformation and will return to their original shape when the actuator 18 is withdrawn.

The combination of the mechanical advantage of the handles 60,74, the threads 52, 80, and the actuator's 18 engagement with the tapered surface 50, results in a large overall mechanical advantage. Thus, a low torsional input on the handles 60, 74 relative to one another to engage the actuator, results in a high radial gripping force on the workpiece. This radial gripping force allows the workpiece to be axially rotated and translated as necessary to carry out the surgical operation. The optional further inclusion of a non-circular engagement tip permits even higher torsional loads to be transmitted to the workpiece.

The locking surgical instrument of the present invention is useful in any surgical application requiring the application of axial and/or torsional forces on a workpiece. It is particularly well suited to applications where such forces must be particularly high and/or where the workpiece must be engaged deep in an implant assembly or narrow surgical wound. The instrument permits reaching into such a narrow space and easily engaging a workpiece with only a small area of the workpiece exposed, without disturbing surrounding structures, and with a simple manipulation of the handles acting to lock the instrument tightly to the workpiece. One use for which the instrument is particularly well suited is shown in FIG. 9 which depicts the locking surgical instrument 10 in use with a rotating hinge knee 90. The rotating hinge knee 90 includes a femoral articulating component 92 and a hinge mechanism 94 held in place by a transverse hinge pin 96. The hinge mechanism 94 includes a hinge post 98 which may be extended by a hinge post extension 100. Both the hinge pin 96 and hinge post extension 100 may be inserted and removed using the instrument 10. The engagement tip 28 is seated in the corresponding female engagement portion of the workpiece. The actuator and body are turned relative to one another to lock the engagement tip 28 in the workpiece, such as by holding the body handle 60 and turning the actuator handle 74. The workpiece may then be turned and axially translated as necessary.

Although embodiments of an instrument and its use have been described and illustrated in detail, it is to be understood that the same is intended by way of illustration and example only and is not to be taken by way of limitation. Accordingly, variations in and modifications to the instrument and its use will be apparent to those of ordinary skill in the art, and the following claims are intended to cover all such modifications and equivalents.

1. A locking surgical instrument for gripping a surgical workpiece, the instrument comprising:
a body including a shaft having a first end, a second end, and an axis therebetween, an engagement tip formed adjacent the second end is receivable by the workpiece, a portion of the engagement tip is divided by one or more slits from the second end toward the first end for a predetermined distance to form a plurality of segments able to be biased radially outwardly to grip the workpiece in force transmitting relationship; and
an actuator engageable with the body such that movement of the actuator biases the segments to grip the workpiece.

2. The instrument of claim 1 wherein the engagement tip engages the workpiece in an axial force transmitting relationship.

3. The instrument of claim 1 wherein the body shaft is axially cannulated from the first end toward the second end such that the cannula extends under the slit portion a predetermined amount and the actuator includes a shaft having a first end, a second end, and an axis therebetween, the shaft being receivable within the cannula for axial translation between an unlocked position in which the actuator second end is spaced from the body shaft second end and a locked position in which the actuator second end is nearer the body shaft second end and biases the segments.

4. The instrument of claim 3 wherein the actuator threadingly engages the body such that turning the actuator causes it to axially translate between the unlocked and locked positions.

5. The instrument of claim 3 wherein the cannula comprises a first diameter spaced from the body shaft second end and a second diameter within the slit portion, the second diameter being smaller than the first diameter such that the actuator is engageable with the second diameter to bias the segments.

6. The instrument of claim 5 wherein the cannula tapers from the first diameter to the second diameter.

7. The instrument of claim 6 wherein the cannula tapers under the slit portion.

8. The instrument of claim 5 wherein the actuator includes a tapered portion near its second end, the actuator tapered portion being engageable with the second diameter of the cannula to bias the segments.

9. The instrument of claim 3 wherein the cannula extends completely through the shaft from the first end to the second end.

10. The instrument of claim 1 wherein the one or more slits each terminate with a circular opening having a radius greater than one-half the width of the slit.

11. The instrument of claim 1 wherein the engagement tip further engages the workpiece in torque transmitting relationship.

12. The instrument of claim 11 wherein the engagement tip is non-circular.

13. The instrument of claim 11 wherein the engagement tip includes flats and vertices, the engagement tip being slit from one flat to another flat such that the vertices are left intact.

14. The instrument of claim 13 wherein the segments are able to be biased by the actuator such that at least one of the vertices is pressed into engagement with the workpiece.

15. The instrument of claim 2 wherein the engagement tip is receivable by a pin of a rotating hinge knee prosthesis to facilitate axial insertion and removal of the pin from the prosthesis.

16. The instrument of claim 15 wherein the engagement tip is receivable by one of a hinge pin and a hinge post extension.

17. A locking surgical instrument for gripping a pin of a knee prosthesis, the instrument comprising:
a body including an elongated shaft having a first end, a second end, and an axis therebetween, a polygonal engagement tip formed adjacent the second end being receivable by a similarly shaped opening on the pin, a portion of the engagement tip being slit from the second end toward the first end for a predetermined distance to divide the tip into a plurality of segments able to be biased radially outwardly to grip the workpiece in axial force transmitting relationship, the body shaft being axially cannulated from the first end toward the second end such that the cannula extends into the slit portion a predetermined amount; and
an actuator including a shaft having a first end, a second end, and an axis therebetween, the shaft being receivable within the cannula for axial translation between an unlocked position in which the actuator second end is spaced from the body shaft second end and a locked position in which the actuator second end is nearer the body shaft second end and biases the segments.

18. The instrument of claim 17 wherein the actuator threadingly engages the body such that turning the actuator relative to the body causes it to axially translate between the unlocked and locked position.

19. A method of gripping a surgical workpiece comprising:
providing an instrument having a shaft with an axis and an engagement tip formed at one end, a portion of the engagement tip being slit to divide the tip into a plurality of segments able to be biased radially outwardly, and an actuator engageable with the body such that movement of the actuator biases the segments outwardly;
inserting the engagement end into an opening in the workpiece; and
moving the actuator to bias the segments outwardly to grip the workpiece in force transmitting relationship.
